(19) United States
(12) Patent Application Publication
(10) Pub. No.: US 2026/0033882 A1
(43) Pub. Date: Feb. 5, 2026

(54) CONNECTION DEVICE AND SURGICAL INSTRUMENT

(71) Applicant: KARL STORZ SE & Co. KG, Tuttlingen (DE)

(72) Inventors: Daniel KÄRCHER, Tuttlingen (DE); Dominik LÄNGLE, Mühlheim (DE); Robin MERZ, Tuttlingen (DE); Sven SCHNEIDER, Tuttlingen (DE)

(21) Appl. No.: 18/998,267

(22) PCT Filed: Jul. 25, 2023
(86) PCT No.: PCT/EP2023/070500

§ 371 Date: Jan. 24, 2025

(30) Foreign Application Priority Data
Jul. 26, 2022 (DE) 10 2022 118 626.1

(51) Int. Cl. A61B 18/12; A61B 18/00; A61B 90/00

(52) U.S. Cl. CPC A61B 18/12; A61B 90/08; A61B 2018/00172; A61B 2018/00202; A61B 2090/0808

(57) ABSTRACT

A connection apparatus for a surgical instrument, having an instrument base, a receiving device arranged at a shaft opening of the instrument base. Further having a first shape-coding dimension and a second shape-coding dimension, and a shaft connection element which is configured to connect an accessory shaft to the instrument base. The shaft connection element is configured as a hollow body. The shaft connection element has a first shape-coding dimension and a second shape-coding dimension. The first and second shape-coding dimensions correspond to the first and second shape-coding dimensions of the receiving device when coupled to the instrument base in an assembled state, and the shaft connection element cannot be assembled with the instrument base if the first and/or second shape-coding dimensions of the shaft connection element and the receiving device do not correspond. The connection apparatus also relates to a surgical instrument with such a connection apparatus.

### CROSS REFERENCE TO RELATED APPLICATIONS

This application is a United States National Phase Application of International Application PCT/EP2023/070500, filed Jul. 25, 2023, and claims the benefit of priority under 35 U.S.C. § 119 of German Application 10 2022 118 626.1, filed Jul. 26, 2022, the entire contents of which are incorporated herein by reference.

### FIELD OF THE INVENTION

The present invention relates to a connection apparatus for a surgical instrument. Furthermore, the invention relates to a surgical instrument comprising such a connection apparatus.

### TECHNICAL BACKGROUND

Surgical instruments are used for different applications. For example, they can be designed as microinvasive medical instruments and comprise a base, for example in the form of a handling device, at the proximal end, a long and usually thin shaft extending from the proximal end to a distal end of the instrument, and a so-called accessory, i.e., a tool or other operating device for gripping, squeezing, coagulating, cutting, punching or other applications at the distal end of the instrument. One or more transmission devices extend in the shaft for transmitting a force and/or a torque from the handling device at the proximal end to the operating device at the distal end. For example, in microinvasive medical instruments having an electrosurgical function, especially in bipolar electrosurgical microinvasive medical instruments, the transmission device is often also involved in the transmission of electrical power from the proximal end to the distal end.

The instruments can often be disassembled into individual components so that different tools, different shafts and other connector elements can be combined. Advantageously, a system with different and therefore versatile possible applications can therefore be provided.

High-quality microinvasive medical instruments are also usually designed to be reusable. In order to simplify cleaning after use, to allow for replacement of a defective component and/or alternative use of different components, it is advantageous for a high-quality microinvasive medical instrument to be disassemblable.

On the one hand, in the case of a disassemblable medical instrument in which the transmission device is also involved in the transmission of electrical power, mechanically disconnectable and safely re-establishable electrical contact with the transmission device should be provided, particularly at the proximal end of the instrument.

Furthermore, components should be combinable with one another in a way that allows them to also be used functionally. This can prevent misuse, damage and the like.

For example, EP 2 892 440 B1 discloses a machining tool set of a surgical, torque-transmitting instrument having at least two different machining tools. Each tool has a distal engagement element that can be inserted and held on the shaft of the instrument by a tool holder in accordance with the key-keyhole principle. Due to different diameters and different lengths, only selected tools can be used. This allows for tool customization that only allows for specific combinations.

DE 19901 398 A1 also discloses a possibility of combining only certain shaft elements with certain handling devices. For this purpose, a specially shaped cut-out is formed in the handling device, into which recess only a shaft element with a corresponding extension can be inserted. In particular, a combination of a RF handling element with an RF-free shaft element can thus be avoided.

A disadvantage of such instruments is that a special connector geometry of the individual components, i.e., in particular the shaft element, the tool or the handling device, is necessary in order to provide tool customization. This requires a plurality of different elements that have to be manufactured differently, which results in complex production. In particular, the costs of the different components that have to be designed are high.

### SUMMARY

Against this background, the object addressed by the present invention is that of providing an improved connection apparatus.

According to the invention, this object is achieved by a connection apparatus having features according to the invention.

Accordingly, the following are provided:
A connection apparatus for a surgical instrument, comprising an instrument base, a receiving device that is arranged at a shaft opening of the instrument base and comprises a first shape-coding dimension and a second shape-coding dimension, a shaft connection element which is designed to connect an accessory shaft to the instrument base, wherein the shaft connection element is designed as a hollow body through which a transmission element guided in the accessory shaft, in particular a pull rod, is passed, wherein the shaft connection element also has a first shape-coding dimension and a second shape-coding dimension, wherein the first and second shape-coding dimensions are dimensioned such that they correspond to the first and second shape-coding dimensions of the receiving device when the shaft connection element is coupled to the instrument base in an assembled state, and such that the shaft connection element cannot be assembled with the instrument base if the first and/or second shape-coding dimensions of the shaft connection element and the receiving device do not correspond.
A surgical instrument, in particular for microinvasive surgery, comprising a connection apparatus, wherein the instrument base can be operated currentless or in a monopolar or bipolar manner, and comprising an accessory that can be operated currentless or in a monopolar or bipolar manner and can be mechanically coupled to the connection apparatus.

The finding forming the basis of the present invention consists in that, by replacing a few elements and keeping as many of the same parts as possible, a surgical instrument can be designed to be individually adaptable to different requirements.

The concept forming the basis of the present invention consists in allowing technically sensible combinations of individual elements and preventing technically unusable combinations by means of a modular structure having adapted coding. A shaft connection element provided as a coupling allows for a combination of at least two components, such as in particular an accessory shaft with an instrument base, in a desired combination and prevents an undesirable combination. Advantageously, the individual components, such as in particular the accessory shaft and the instrument base, thus do not have to have the coding themselves. Rather, the coding is provided by the design of the shaft connection element. Said element comprises a first shape-coding dimension and a second shape-coding dimension which correspond to a first and second shape-coding dimension of the receiving device when the shaft connection element is coupled to the instrument base in an assembled state. In this way, non-functional connections between elements can be prevented if the individual elements are specifically designed for a surgical instrument operated in a monopolar or bipolar manner or currentless. Misuse, damage to individual elements and the like are blocked and thus prevented by unauthorized combinations. This is to ensure that, in the case of a plurality of different systems, such as a surgical instrument operated in a monopolar or bipolar manner or currentless, only those components that are intended to be combined can be combined with one another.

In an example embodiment, the shaft connection element cannot be assembled with the instrument base if the first or second shape-coding dimension of the shaft connection element does not correspond to the corresponding first or second shape-coding dimension of the receiving device. This is the case, for example, if the shaft connection element is either too wide or too long in relation to the receiving device. In a further embodiment, the first and second shape-coding dimensions of the shaft connection element may not correspond to the corresponding first and second shape-coding dimensions of the receiving device, in which case, of course, the elements then cannot be connected to one another either.

In addition, such a shaft connection element also allows for backward compatibility with existing systems. This can make it possible to combine an existing instrument base with a newly designed accessory shaft.

Furthermore, in this way it is advantageously possible to set up a so-called matrix of all the possible combinations, whereby new developments, which are to be combined with the surgical instrument in the future, having corresponding new shaft connection elements can be easily added. Likewise, individual elements, such as the accessory shaft or the instrument base, can be used for more than one surgical instrument, since coding and compatibility are simultaneously provided by the shaft connection element.

The instrument base can have different designs, for example, in the case of a manually actuable surgical instrument, it can comprise a handle element or in the case of a robot-operated instrument, it can comprise a robot holder. In particular, the instrument base itself can be designed as a handle and comprise at least the shaft opening. Furthermore, a thumb ring element or the like can be arranged on the instrument base. In particular, further elements, such as a radio-frequency connector (RF connector), in particular in the form of an RF plug element, can be connected to the instrument base. For this purpose, the instrument base can have additional receiving openings.

A shaft opening is to be understood in particular as a receptacle for the accessory shaft, which is initially designed to receive the shaft connection element. Latching means for fixing the shaft connection element to the accessory shaft can be arranged at the shaft opening.

A shape-coding dimension is to be understood in particular to mean a predetermined geometric dimension of a configuration which geometrically allows or geometrically prevents a connection between the shaft connection element and the instrument base. In particular, the geometric dimensions of the shaft connection element and the instrument base are therefore coordinated or correspond with one another if they are intended to be connectable to one another, and do not fit together or cannot be assembled if they are not intended to be connectable to one another.

In particular, two shape-coding dimensions can be used to code a plurality of sub-combinations in a predetermined manner so that only parts that fit together or are allowed to be combined can be assembled with one another. In particular, it is sufficient if one of the two shape-coding dimensions for the shaft connection element and the receiving device does not match or correspond in order to prevent assembly. On the other hand, sub-combinations are in particular possible in which one of the two shape-coding dimensions is not the same, but a corresponding or mountable combination is still provided, for example if an external dimension to be received is smaller than an internal dimension to be received. However, a combination that cannot be assembled or does not correspond arises in particular if, for example, in one of the two shape-coding dimensions an external dimension to be received is larger than an internal dimension to be received. In this way, in particular, a plurality of different permissible combinations of first and second shape-coding dimensions can be permitted and only the assembly of individual impermissible combinations can be excluded.

Advantageous embodiments and developments are shown in the further dependent claims and in the description with reference to the figures in the drawing.

According to an advantageous embodiment, the smallest inner diameter can be formed as the first shape-coding dimension of the receiving device and the largest outer diameter can be formed as the first shape-coding dimension of the shaft connection element. It is advantageous to understand the smallest and largest outer diameters as the smallest and largest possible outer diameters. This makes it possible, provided the combination is permissible, to assemble the shaft connection element with the accessory shaft on or in the instrument base.

According to a development, the shaft connection element may not be completely insertable into the receiving device if the first shape-coding dimension of the shaft connection element is larger than the first shape-coding dimension of the receiving device. This can prevent impermissible combinations from being connectable to one another. The first shape-coding dimension of the receiving device can therefore form the largest possible length or width that the first shape-coding dimension of the shaft connection element can have in order to establish a connection. If the first shape-coding dimension of the shaft connection element is longer or wider than this, the shaft connection element cannot be assembled with the instrument base, making the surgical instrument unusable.

According to one embodiment, the second shape-coding dimension of the receiving device can be a distance provided in the assembled state between a stop provided in the instrument base and a latching means that is integrated into the receiving device, and the second shape-coding dimension of the shaft connection element can be a length provided between an end corresponding to the stop and a projection corresponding to the latching means. This advantageously allows the shaft connection element and the instrument base to be axially fixed with respect to one another, as described in the previous paragraph. In particular, in conjunction with a first shape-coding dimension of the receiving device, the stop can act as a largest possible permissible length, wherein the latching means only engage when the length of the shaft connection element is adapted to the length of the receiving device or does not exceed it.

According to an advantageous embodiment, the shaft connection element may not be completely insertable into the receiving device if the second shape-coding dimension of the shaft connection element is larger than the second shape-coding dimension of the receiving device. In such a case, for example, a functionally undesirable or technically impermissible combination is provided, which accordingly cannot be geometrically fixed or assembled, and therefore it is not possible for the shaft connection element to engage or be fixed in the instrument base.

According to a preferred embodiment, the receiving device can be designed as a rotary wheel arrangement which is designed for the rotational manipulation of the accessory shaft. The latching means can be integrated into the rotary wheel arrangement. When the latching means is activated, the instrument base can be released from the accessory shaft using the tool. Therefore, the receiving device can simultaneously fix and control the accessory shaft.

According to a particularly preferred embodiment, the latching means integrated in the receiving device can latch with the projection of the shaft connection element for axially securing the accessory shaft in the longitudinal direction with a degree of rotational freedom. Advantageously, the projection is therefore designed as a circumferential projection that runs around the entire shaft connection element. The latching means can be integrated as a movable pin element in the rotary wheel arrangement. As a result, the latching means is always in contact with the projection in the axially secured state, even if the rotary wheel arrangement is rotated about the longitudinal axis of the accessory shaft.

According to an advantageous embodiment, different first and/or second and/or third shape-coding dimensions can be provided for an accessory and an instrument base having a currentless connection or a current-carrying monopolar RF connection to those provided for an accessory and an instrument base having a bipolar RF connection. This can advantageously prevent an undesirable or technically impermissible combination, for example of elements not designed for current-carrying operation with a current-carrying instrument base.

According to a development, the first shape-coding dimensions can be designed such that a shaft connection element of an accessory shaft of a bipolar accessory cannot be inserted into a receiving device for currentless or monopolar connection, in particular as a result of an outer diameter of the shaft connection element that is larger than the inner diameter of the receiving device. In this way, a coded assignment of technically reasonable combinations of the elements can be created. Alternatively or additionally, the first shape-coding dimensions can be designed such that a shaft connection element of an accessory shaft of a monopolar accessory part can be inserted into a receiving device for currentless connection. This is possible because an element designed for current-carrying applications can also be used for currentless applications. Although such a combination would not be technically preferred, it would still be permissible because the instrument base would be operated without power, and therefore no damage would occur when an accessory designed to carry current were connected and a surgical instrument designed in this way would still function when operated currentless.

According to one embodiment, the second shape-coding dimensions can be designed such that a shaft connection element of an accessory shaft of a currentless accessory cannot be completely inserted into a receiving device for monopolar or bipolar connection, in particular as a result of a length between a proximal end and a projection of the shaft connection element provided for latching purposes, which is larger than a distance provided between a stop provided in the instrument base and a latching means that is integrated in the receiving device. In this way, damage to the elements and in particular a short circuit can be avoided, since the instrument base designed for bipolar or monopolar application cannot be combined with shaft elements that are only designed for currentless applications.

In an advantageous embodiment, the instrument base can have a guide element, in particular one which is integrally formed with the stop, which is designed to guide and/or electrically contact the transmission element guided in the accessory shaft, in particular a pull rod, the guide element having a through-opening with a third shape-coding dimension and the transmission element also being designed to have a third shape-coding dimension, the third shape-coding dimensions of the through-opening being dimensioned such that they correspond to the third shape-coding dimension of the transmission element when the transmission element is guided through the through-opening, and such that the transmission element cannot be inserted into the through-opening if the third shape-coding dimensions of the through-opening and of the transmission element do not correspond. The third shape-coding dimension is formed, for example, as a circular opening through which the transmission element, which is also formed having a circular cross section, can be guided. The opening is advantageously larger than the largest diameter of the transmission element, in particular larger than a pull rod head of the pull rod element. The guide element can be part of a connector body which is arranged in a radio-frequency connector and/or is designed as a plug element which can be detached from the instrument base. The pull rod element can be passed through the guide element and hooked onto an actuating element, in particular a thumb ring element, which can also be fixed to the instrument base.

According to an advantageous embodiment, the transmission element can be designed to actuate an accessory insert that can be coupled to the distal end of the accessory shaft, for example a forceps, scissors or clamp insert, wherein the shaft connection element is arranged at a proximal end of the accessory shaft and the guide element can be arranged so as to be proximally connected thereto in an assembled state. The accessory insert therefore forms a tool. The guide element can form the stop of the second shape-coding dimension of the receiving device.

According to an advantageous embodiment, the guide element can be designed as part of a monopolar or bipolar radio-frequency connector, in particular as a pre-assembled plug-in module for modular installation and removal in and from an instrument base. In this way, a modular system can be provided that is characterized by the highest possible number of pre-assembled components. Furthermore, such a radio-frequency connector can be used to align the connector and the shaft connection element with the accessory shaft in the instrument base.

According to an advantageous embodiment, the third shape-coding dimensions can be designed such that a transmission element of a monopolar accessory cannot be inserted into a through-opening of a guide element for bipolar connection, in particular as a result of an outer diameter of the transmission element which is larger than the diameter of the opening. For example, in such a case the pull rod head has a larger diameter than the diameter of the opening. This can prevent a connection between the shaft connection element and the instrument base.

According to a development, the instrument base can be designed as a handle. In particular, the instrument base is designed as a handle with an actuating element for manual actuation. The actuating element can, for example, have a finger ring and a thumb ring, wherein the thumb ring is coupled to the transmission element, in particular to the pull rod head of the pull rod. This means that the accessory coupled to the transmission element, which is preferably designed as a clamp or scissors, can be activated by the handle. In other embodiments, it would also be possible to couple the transmission element to a finder ring.

In a further embodiment, the instrument base can be designed as a manipulator coupling or as a robot holder for actuating the accessory that is coupled thereto via the accessory shaft and the transmission element by means of an actuator. This also makes it possible to use the surgical instrument in the field of robotic surgery.

The above embodiments and developments can be combined with each other as desired, if appropriate. Further possible embodiments, developments, and implementations of the invention also include combinations, which are not explicitly mentioned, of features of the invention described above or below with respect to the exemplary embodiments. In particular, a person skilled in the art will also add individual aspects as improvements or additions to the particular basic form of the present invention.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is an embodiment of a shaft connection element of a connection apparatus;

FIG. 2 is another embodiment of a shaft connection element of a connection apparatus;

FIG. 3 is an embodiment of a receiving device;

FIG. 4 is the receiving device from FIG. 3 with the shaft connection element inserted;

FIG. 5 is the receiving device from FIG. 3 with the shaft connection element and pull rod inserted;

FIG. 6 is a connection apparatus with mismatching elements;

FIG. 7 is a connection apparatus with mismatching elements;

FIG. 8 is a connection apparatus with mismatching elements;

FIG. 9 is an embodiment of a connector arrangement;

FIG. 10 is a further view of the embodiment from FIG. 9;

FIG. 11 is an embodiment of a connector body;

FIG. 12 is a further view of the embodiment from FIG. 11;

FIG. 13 is an embodiment of an instrument base with a radio-frequency connector;

FIG. 14 is a detailed view of the embodiment according to FIG. 13 with a detailed view of an embodiment of the radio-frequency connector;

FIG. 15 is an embodiment of a coupling of the pull rod head with the handle element;

FIG. 16 is a detailed view of an embodiment of a connection apparatus;

FIG. 17 is an embodiment of a surgical instrument;

FIG. 18 is a detailed view of another embodiment of a connection apparatus; and

FIG. 19 is an embodiment of a shaft connection element arranged on an accessory shaft.

The accompanying figures of the drawing are intended to provide a further understanding of the embodiments of the invention. They illustrate embodiments and, in conjunction with the description, serve to explain principles and concepts of the invention. Other embodiments and many of the advantages mentioned are shown in the drawings. The elements in the drawings are not necessarily shown to scale.

In the figures in the drawing, like, functionally like and identically acting elements, features and components are each provided with the same reference signs, unless otherwise specified.

### DESCRIPTION OF PREFERRED EMBODIMENTS

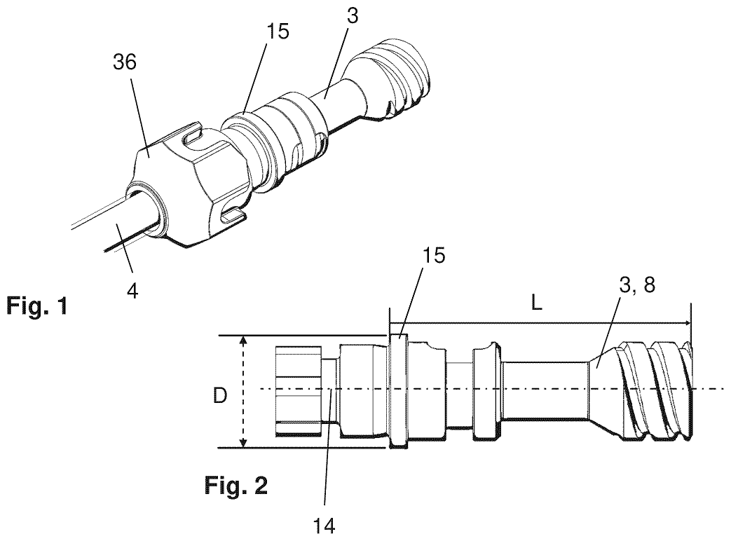

Referring to the drawings, FIG. 1 shows an embodiment of a shaft connection element 3 of a connection apparatus 1. The shaft connection element 3 is designed to connect an accessory shaft 4 to an instrument base 5 (not shown). The shaft connection element 3 is designed as a hollow body 8 through which a transmission element guided in the accessory shaft 4, such as a pull rod (not shown), is passed. The shaft connection element 3 has a first shape-coding dimension and a second shape-coding dimension. In the embodiment shown in FIG. 2, these are characterized by a diameter D and a length L.

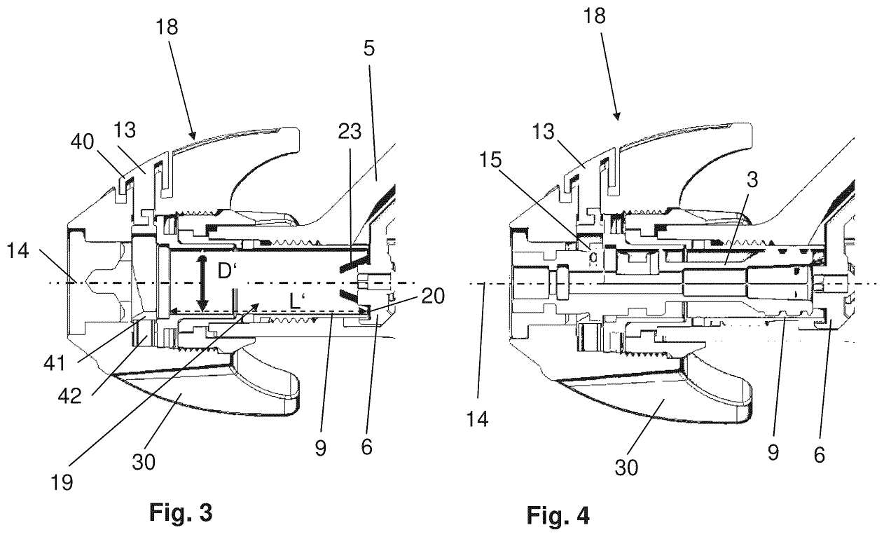

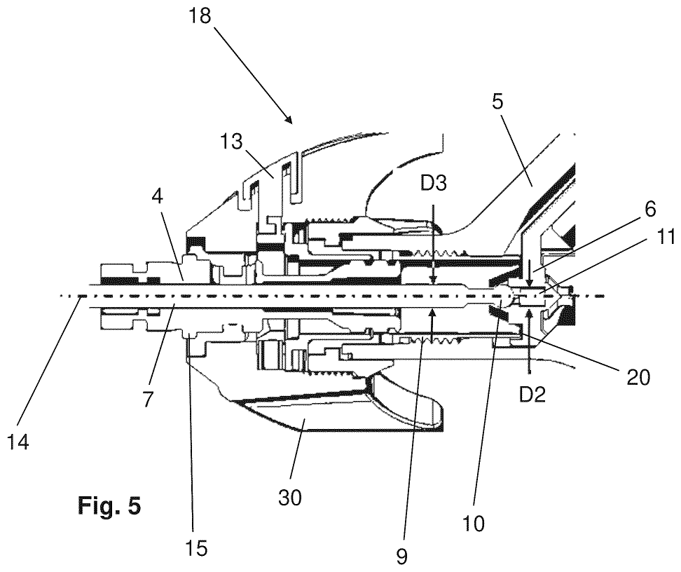

The first and the second shape-coding dimensions are dimensioned such that they correspond to a first and a second shape-coding dimension of a receiving device 18 of an instrument base 5, shown for example in FIG. 3, when the shaft connection element 3 and the instrument base 5 belong to a common system and are couplable in an assembled state. However, if the shaft connection element 3 and the instrument base 5 do not correspond, they cannot be inserted into one another and cannot be coupled. Coupling or fixing the shaft connection element 3 and the instrument base 5 or the receiving device 18 to one another can be provided, for example, by a projection 15 on the shaft connection element 3; this is shown in detail in FIGS. 5 to 8.

As can be seen in FIG. 1, there is a cover 37 with a so-called 4×90° geometry on the accessory shaft. This means that four recesses are arranged along the circumference of the cover, which can engage in a counter contour on a rotary wheel arrangement 30, shown in FIG. 3 et seq. and FIG. 16.

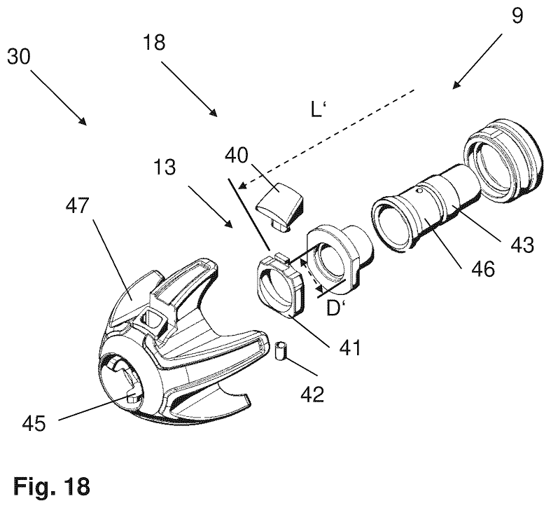

FIG. 3 shows an embodiment of a receiving device 18. The receiving device 18 is arranged at a shaft opening 19 of an instrument base 5 and has a first and second shape-coding dimension. A detailed view of the shape codings is shown, for example, in FIG. 18. A first shape-coding dimension can be, for example, the smallest inner diameter D′ and a second shape-coding dimension can be the shortest distance L′ provided between a latching means 13 and a stop 20. Consequently, for a corresponding connection, the dimensions D and L must match or correspond to the dimensions D′ and L′ so that a shaft connection element 3 can be assembled with an accessory shaft 4 in an instrument base 5.

FIG. 4 shows the receiving device from FIG. 3 with the shaft connection element 3 inserted.

The latching means 13 is designed as a spring-loaded catch integrated into the rotary wheel arrangement 30, which catch is designed to engage with the shaft connection element 3. For example, the catch has a through-opening through which the shaft connection element 3 can pass. An outer head portion 40 of the catch is provided as an actuating portion, while a spring 42 preloading the catch is arranged on a foot portion 41.

It can be seen that the shaft connection element 3 is too long for the receiving device 18 and so the latching means 13 cannot latch with the projection 15. It is therefore not possible to secure the shaft connection element 3 in the longitudinal direction 14 of the surgical instrument. A connection between the shaft connection element 3 and the receiving device 18 is therefore not possible in this embodiment. This may be the case, for example, if incompatible elements of currentless and current-carrying surgical instruments were connected by mistake.

FIG. 5 shows the receiving device from FIG. 4 with the shaft connection element 3 and pull rod 7 inserted. The pull rod 7 touches the through-opening 11, but cannot pass therethrough. Consequently, a diameter D2 of the through-opening 11 represents a third shape-coding dimension of the receiving device 18, and a diameter D3 of the pull rod 7, which is designed as a transmission element for establishing electrical contact, represents a third shape-coding dimension of the shaft connection element 3.

The diameter of the transmission element, i.e., in this case the diameter D3 of the pull rod 7 or the pull rod head 10, can be, for example, between 2 mm and 2.5 mm. The diameter D2 of the through-opening 11 can therefore be, for example, between 2.1 mm and 2.3 mm, so that a pull rod head 10 with a larger diameter cannot be guided through the through-opening 11. In this way, coding can be provided for different instrument systems. Advantageously, the pull rod head 10 which is too large is the first to touch the stop 20 or the through-opening 11 which is too small, so that a user can detect an incorrect combination very early on from the shaft connection element 3 protruding. In particular, damage caused by a with too much force applied can therefore be avoided.

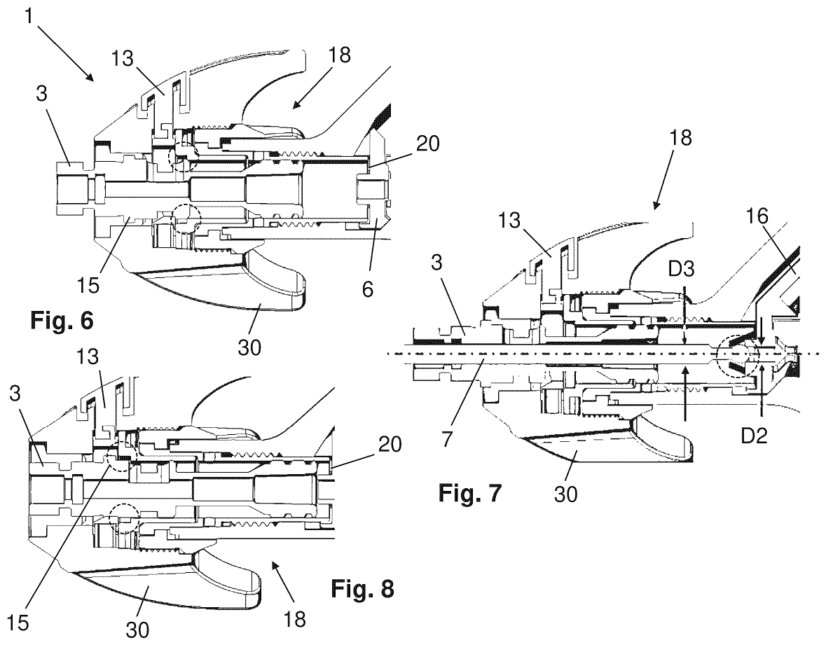

FIG. 6 also shows a connection apparatus 1 with mismatching elements. The diameter of the shaft connection element 3 is larger than the diameter of the receiving device 18 and therefore cannot engage in the diameter of the receiving device 18. For example, the diameter may be approximately 0.1 mm to 1 mm larger, preferably 0.1 mm to 0.5 mm, preferably 0.1 mm to 0.3 mm, for example 0.2 mm. In this case, the diameter of the shaft connection element 3 could be, for example, 8 mm and thus not engage in the diameter of the receiving device 18, which is 7.8 mm in size. The blocking regions are shown by dashed circles.

FIG. 7 shows another connection apparatus 1 with mismatching elements. In this case, the diameter of the pull rod head 10 is too large for the diameter of the through-opening 11. This corresponds to the case in FIG. 5.

FIG. 8 shows another connection apparatus 1 with mismatching elements. The shaft connection element 3 can, for example, be at least 1 mm, in particular 1 mm to 5 mm, preferably 1 mm to 3 mm, for example 2 mm, longer than the distance between the stop 20 and the latching means 13 of the receiving device 18. In this case, it can, for example, have a length of 26 mm, wherein the distance between the stop 20 and the latching means 13 of the receiving device 18 is only 24 mm. Consequently, the shaft connection element 3 cannot be fixed so as to latch with the receiving device 18.

Each of the embodiments in FIGS. 6 to 8 represent different shape-coding dimensions. For example, FIG. 6 shows a first shape-coding dimension and FIG. 8 shows a second shape-coding dimension. The situation in FIG. 7 may represent a third shape-coding dimension.

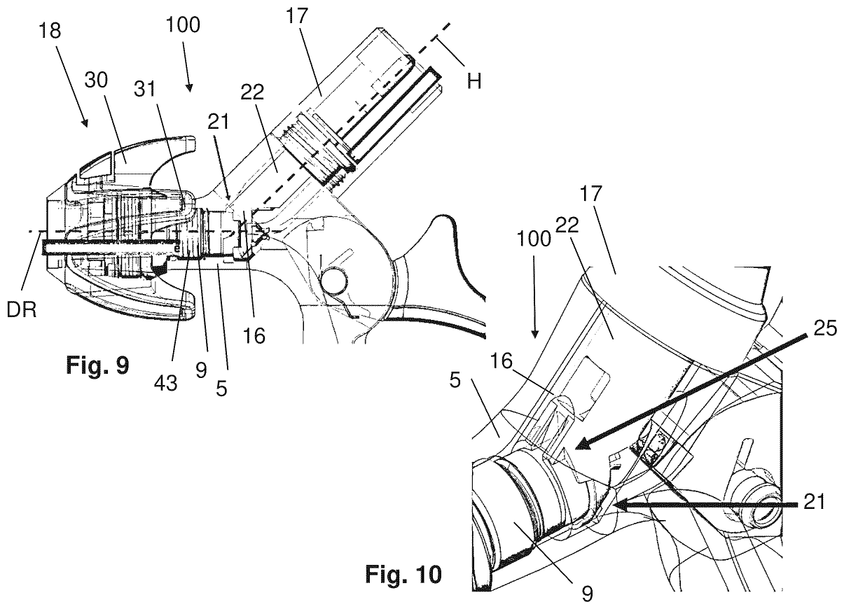

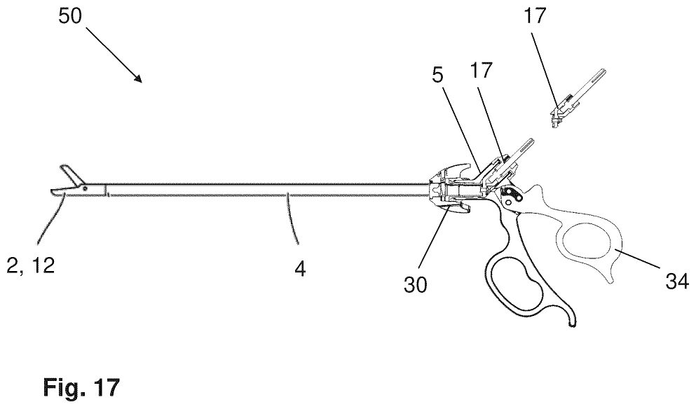

FIG. 9 and FIG. 10 show an embodiment of a connector arrangement 100. The instrument base 5 is designed to activate and/or operate a radio-frequency tool 2, which is shown in FIG. 17, for example.

A radio-frequency connector 17 assembled in the instrument base has a connector body 16. The connector body 16 has an alignment portion 22 which serves for the predetermined alignment of the RF connector 17 on a connector axis H within the instrument base 5. Furthermore, the connector body has a locking portion 21 for connection to a rotary wheel arrangement 30 of the surgical instrument at a predetermined angle of a rotary wheel axis DR with respect to the connector axis H. The rotary wheel arrangement 30 has a coupling portion 31 for coupling to the connector body 16. By means of the coupling, the rotary wheel arrangement 30 can be aligned in the predetermined rotary wheel axis RW with respect to the connector axis H in the instrument base 5.

For rotational alignment of the connector body 16 and the rotary wheel arrangement 30, at least one engagement element 25 (shown in FIG. 10) is arranged in the instrument base 5, which can be connected to the coupling element 31 in a manner in which it engages therewith or is coupled thereto.

The receiving device 18 can be pre-assembled. For example, the rotary wheel arrangement 30 can be manufactured so as to be pre-assembled with corresponding receiving elements 9, which in particular have the shape-coding dimensions, as a specially coded design. During assembly, the rotary wheel arrangement 30 and the RF connector 17 can thus be inserted into the instrument base 5 from different directions. The predetermined axes H and RW or their predetermined angles allow the coupling to be formed without seeing it.

By means of a locking geometry 27, the two assemblies can finally be connected to one another in the instrument base 5 without any further aids. At the same time, the RF connector 17 and the receiving device 18, i.e., the rotary wheel arrangement 30, can be aligned without any aids. The alignment ensures that the RF contacts are concentric with the inserted accessory shaft 4, the axis of which is defined by the rotary wheel arrangement 30. To a certain extent, tolerance compensation can also be achieved for the components.

Advantageously, the axes H and RW are consistent and are arranged at a predefined angle to one another to ensure proper functioning of the surgical instrument. In particular, this avoids misalignment, which could lead to contact problems during current transmission or to grinding or even blocking of the pull rod 7.

For assembly, for example, the rotary wheel arrangement 30 can first be partially, in particular minimally, screwed into the instrument base 5 via a thread 43. The RF connector 17 can then be inserted into the instrument base 5. Subsequently, the rotary wheel arrangement 30 is advantageously completely screwed into the instrument base 5. When the RF connector 17 comes into contact with the receiving device 18 (with the rotary wheel arrangement 30), they are aligned with one another. Since RF contact elements 23 are pre-assembled on the RF connector 17, any axial misalignments that may occur can be tolerated or compensated for thereby. Pre-assembly still allows complex and elaborate designs to be implemented, which can nevertheless be assembled relatively easily due to easy accessibility and/or can be designed with a comparatively small number of connection points.

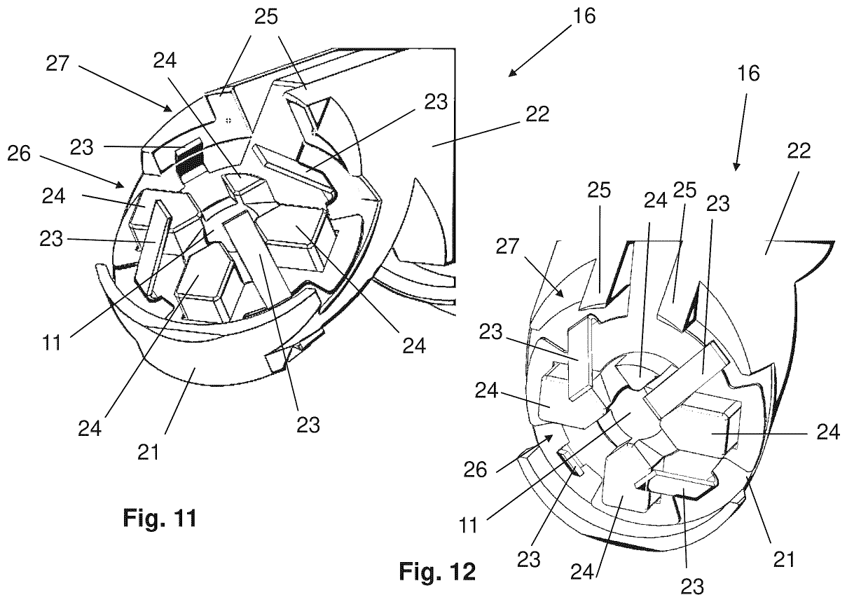

FIG. 11 and FIG. 12 show an embodiment of a connector body 16 of an RF connector 17. The connector body 16 has a locking portion 21 and an alignment portion 22. In the region of the locking portion 21, a protective geometry 26 is provided to protect the RF contact arrangement. Consequently, the protective geometry 26 can protect the RF contact elements 23 from damage when inserting the RF tool 2, in particular when inserting the shaft and/or the pull rod.

The protective geometry 26 can be formed by a plurality of alignment projections 24, wherein the alignment projections 24 and the RF contact elements 23 are arranged adjacently to one another in an alternating fashion. Consequently, the contact elements 23 are each placed in a kind of gap between the alignment projections 24. For example, if an incompatible accessory shaft with an incompatible pull rod head 10 is inserted into the instrument base 5, it will touch the surfaces of the alignment projections 24, thereby preventing damage to the RF contact elements 23. Furthermore, it is possible that the RF contact elements 23 are deflected into the gaps between the alignment projections 24 in the event of an overload so that they are not destroyed or rendered unusable by plastic deformation. Furthermore, the protective geometry 26 can ensure the alignment and position of the RF contact elements 24.

The alignment projections 24, together with the engagement element 25, further form a locking geometry 27 which is designed for rotation-proof engagement with the rotary wheel arrangement 30. In particular, the receiving device 18 can have not only a rotary wheel arrangement 30, but also at least one receiving element 9, which can also come into contact with the locking geometry 27 and have a fixing effect. Such a receiving element 9 can be designed in several parts and is shown, for example, in FIG. 18 in an example embodiment.

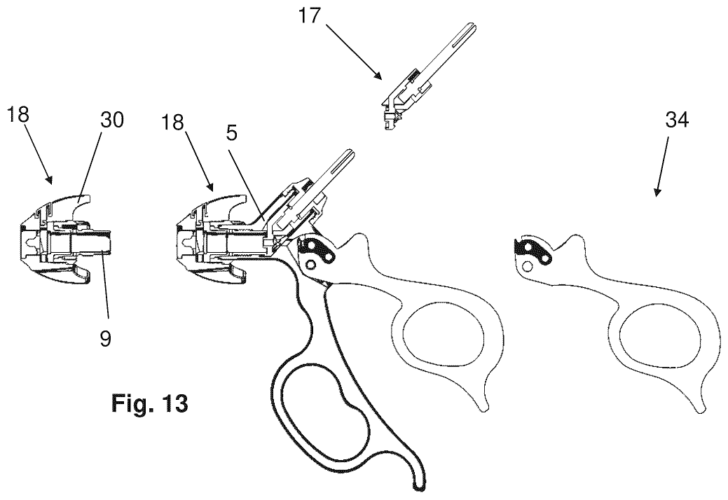

FIG. 13 shows an embodiment of an instrument base 5 with an RF connector 17, wherein the assemblies assembled with the instrument base 5 are additionally shown individually.

The RF connector 17 is designed as a pre-assembled plug-in module for modular installation and removal in and from the instrument base 5. For this purpose, the RF contact elements 23 are also pre-assembled on the plug-in module. The plug-in module can thus be inserted into the instrument base 5 along a connector axis H and assembled.

Furthermore, a removable thumb ring 34 is arranged on the instrument base.

The rotary wheel arrangement is mounted or screwed in along the axis of rotation RW.

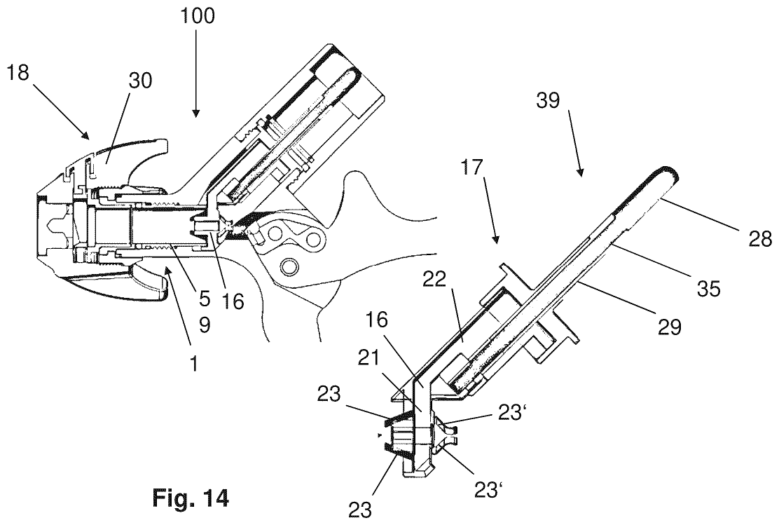

FIG. 14 is a detailed view of the radio-frequency connector 17, once in the assembled position and once on its own. The connector body 16 has a plug portion 39 which can be connected in the direction of the connector axis H and to which the RF contact arrangement is conductively coupled. The plug portion 39 can have at least one plug pin 28 and an RF contact element 23 which is made from a continuous bent sheet metal part.

Further RF contact elements 23′ which serve to contact the pull rod 7 are arranged in the region of the through-opening 11, in the illustration in a side facing away from the locking portion 21. One RF contact element 23 and one RF contact element 23′ can be made from a continuous bent sheet metal part each time. The plug portion 39 can also have a solid steel core in the interior, which, for example, forms a second plug pin 29 and is welded to the RF contact element 23′.

The receiving device 18 and the RF connector 17 can be inserted into the instrument base 5 from different sides, for example as shown in FIG. 13. Both assemblies are structurally designed in such a way that they can function with a minimal number of components and can be functionally and modularly assembled or replaced. For example, the steel core protects against damage and is therefore particularly robust during application. Advantageously, the contact resistances within the RF connector 17 are produced by an integral connection, for example by welding, and are therefore very low-resistance. The components 17 and 18 can therefore be pre-assembled completely independently and then inserted directly into the instrument base 5 and used within the sense of “plug-and-play” assembly.

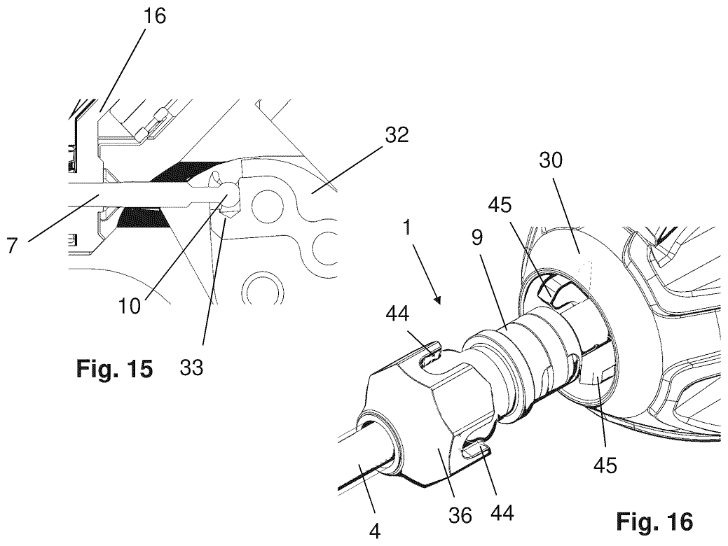

FIG. 15 shows an embodiment of a coupling of the pull rod head 10 to an actuating part of the instrument base 5. The pull rod 7 comprising the pull rod head 10 is passed through the through-opening 11 in the connector body 16 and guided to a coupling region 33 of the actuating element, which is designed here as a thumb ring 34. The thumb ring 34 forms a movable handle leg and has a ball holder in the coupling region 33. This ball holder can be integrated in a form-fitting manner during the manufacture of the thumb ring 34, in particular by injection molding. The pull rod head 10 can be received in this ball holder and thus transmit a movement in the proximal to distal direction, in particular for opening and closing the accessory insert 12, such as a scissor tool.

FIG. 16 is a detailed view of an embodiment of a connection apparatus 1.

As explained with reference to FIG. 1, a cover 37 with a so-called 4×90° geometry is arranged on the accessory shaft. The four recesses 44 provided for this purpose along the circumference of the cover 37 can engage in the counter contour of the rotary wheel arrangement 30. The counter contour of the rotary wheel arrangement 30 is formed by protruding pins 45 corresponding to the recesses 44; two of four pins 45 can be seen in the view.

FIG. 17 shows an embodiment of a surgical instrument 50.

The surgical instrument 50 has an accessory insert 12 in the form of an RF tool 2 at the distal end. The accessory shaft 7 extends proximally from the distal end to the instrument base 5. There, the accessory shaft 4 is received in the instrument base 5 via the rotary wheel arrangement 30 as the receiving device.

Furthermore, an RF connector 17 is inserted into the instrument base 5 in a different axis. The RF connector 17 is attached at an angle of 45° to the top of the instrument base 5 and thus leads the radio-frequency cable away from the operating field.

Furthermore, an actuating element, here the thumb ring 34, is assembled on the side of the instrument base 5 that is opposite the accessory shaft 4. When the actuating element is positioned horizontally, i.e., when the thumb ring 34 is positioned horizontally here, the accessory shaft can be decoupled from the actuating element, in particular the pull rod head can then be removed from the ball head holder.

The rotary wheel arrangement 30 comprises the latching means 13 described above. When the actuating element is positioned horizontally, pressing a button on the head portion 41 of the latching means 13 is sufficient to separate the accessory shaft 4 from the actuating element. Then, for disassembly, the rotary wheel arrangement 30 and the RF connector 17 can also be removed in a modular manner from the instrument base 5, in this case by unscrewing it and by pulling it out, respectively.

FIG. 18 shows a further embodiment of a connection apparatus 1 with a detailed view of the receiving device 18.

In this embodiment, the receiving device 18 of the rotary wheel arrangement 30 has a receiving element 9 which is formed in several parts. Thus, the receiving device 18 can be formed from a plurality of sleeve-shaped elements which are fixed together. Individual elements can rotate within the instrument base 5 in the assembled state, others can be fixed thereto. In particular, the sleeve-shaped element 46 shown in the middle, which has the largest extent in the longitudinal direction 14, can be screwed to the instrument base 5 via the thread 43. The other sleeve-shaped elements rotate when the rotary wheel of the rotary wheel arrangement 30 moves.

On the left side of the illustration, the rotary wheel 47 and the latching means 13 of the rotary wheel arrangement 30 can also be seen. In the exploded view shown, the latching means 13 is shown detached from the rotary wheel 47.

In the distal opening of the rotary wheel arrangement 30, the pin elements 45 integrated therein can be seen, which can engage in the recesses 44 of the cover 36, which is shown in FIG. 1.

In this embodiment, the first and second shape-coding dimensions are formed by the plurality of elements in the assembled state. These are schematically represented by a maximum diameter D′ and a maximum length L′ that is indicated by dashed lines and is, of course, adapted in the installed state.

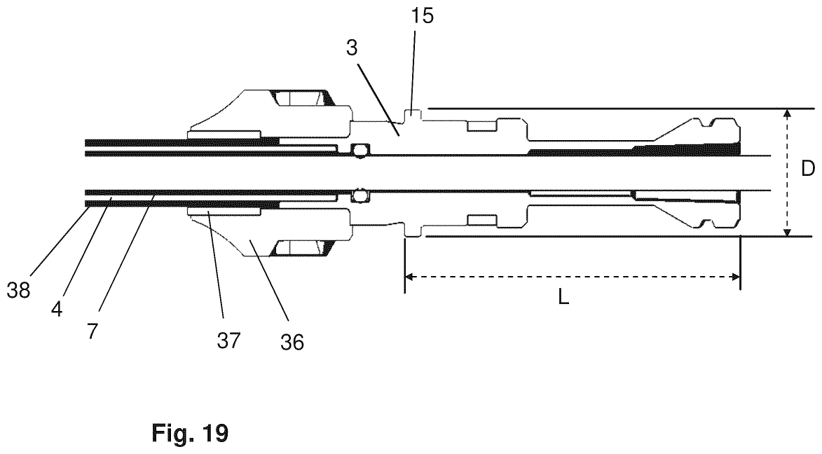

FIG. 19 shows an embodiment of a shaft connection element 3 arranged on an accessory shaft 4.

In the connection apparatus shown here, the pull rod 7 extends in the accessory shaft 4. An insulating coating 38 is provided on the outside of the accessory shaft 4, for example a Halar (ECTFE) coating.

A seal 37 is provided between the accessory shaft 4 and the cover 36, for example in the form of a sealing lip. The projection 15 can be seen on the shaft connection element 3 and represents a measure of the first and second shape-coding dimensions. These are characterized by the diameter D and the length L.

The seal 37, the cover 36 and the shaft connection element 3 can form a two-component injection molded insert part.

Although the present invention has been fully described above with reference to preferred exemplary embodiments, it is not limited thereto, but can be modified in a variety of ways. In particular, the shaft connection element 3 can therefore have a geometry that differs from that shown. Furthermore, the receiving device 18 can have a geometry that differs from the embodiment shown, in particular by differently shaped individual elements, such as individually shaped receiving elements 9. Likewise, the coupling between the rotary wheel arrangement 30 and the cover 36 can have a different design.

While specific embodiments of the invention have been shown and described in detail to illustrate the application of the principles of the invention, it will be understood that the invention may be embodied otherwise without departing from such principles.

1. A connection apparatus for a surgical instrument, the connection apparatus comprising:
an instrument base,
a receiving device that is arranged at a shaft opening of the instrument base and comprises a first shape-coding dimension and a second shape-coding dimension, and
a shaft connection element which is configured to connect an accessory shaft to the instrument base, wherein the shaft connection element is configured as a hollow body through which a transmission element is guided in the accessory shaft, is passed, wherein the shaft connection element also has a first shape-coding dimension and a second shape-coding dimension, wherein the first and second shape-coding dimensions are dimensioned such that they correspond to the first and second shape-coding dimensions of the receiving device when the shaft connection element is coupled to the instrument base in an assembled state, and such that the shaft connection element cannot be assembled with the instrument base if the first and/or second shape-coding dimensions of the shaft connection element and the receiving device do not correspond.

2. The connection apparatus according to claim 1, wherein the smallest inner diameter is formed as the first shape-coding dimension of the receiving device and the largest outer diameter is formed as the first shape-coding dimension of the shaft connection element.

3. The connection apparatus according to claim 1, wherein the shaft connection element cannot be completely inserted into the receiving device if the first shape-coding dimension of the shaft connection element is larger than the first shape-coding dimension of the receiving device.

4. The connection apparatus according to claim 1, wherein the second shape-coding dimension of the receiving device is a distance provided in the assembled state between a stop provided in the instrument base and a latching means that is integrated into the receiving device, and the second shape-coding dimension of the shaft connection element is a length provided between an end corresponding to the stop and a projection corresponding to the latching means.

5. The connection apparatus according to claim 1, wherein the shaft connection element cannot be completely inserted into the receiving device if the second shape-coding dimension of the shaft connection element is larger than the second shape-coding dimension of the receiving device.

6. The connection apparatus according to claim 1, wherein the receiving device is configured as a rotary wheel arrangement for the rotational manipulation of the accessory shaft.

7. The connection apparatus according to claim 4, wherein the latching means integrated in the receiving device is configured to latch with the projection of the shaft connection element for axially securing the accessory shaft in the longitudinal direction with a degree of rotational freedom.

8. The connection apparatus according to claim 1, wherein different first and/or second and/or third shape-coding dimensions are provided for an accessory and an instrument base having a currentless connection or a current-carrying monopolar RF connection to those provided for an accessory and an instrument base having a bipolar RF connection.

9. The connection apparatus according to claim 8, wherein the first shape-coding dimensions are configured such that a shaft connection element of an accessory shaft of a bipolar accessory cannot be inserted into a receiving device for currentless or monopolar connection.

10. The connection apparatus according to claim 8, wherein the second shape-coding dimensions are configured such that a shaft connection element of an accessory shaft of a currentless accessory cannot be completely inserted into a receiving device for monopolar or bipolar connection.

11. The connection apparatus according to claim 1, wherein the instrument base has a guide element, which is integrally formed with the stop, which is configured to guide and/or electrically contact the transmission element guided in the accessory shaft, wherein the guide element has a through-opening with a third shape-coding dimension and the transmission element is also configured to have a third shape-coding dimension, wherein the third shape-coding dimensions of the opening are dimensioned to correspond to the third shape-coding dimension of the transmission element when the transmission element is guided through the through-opening, and such that the transmission element cannot be inserted into the through-opening if the third shape-coding dimensions of the through-opening and of the transmission element do not correspond.

12. The connection apparatus according to claim 11, wherein the transmission element is configured to actuate an accessory insert that can be coupled to the distal end of the accessory shaft, for example wherein the shaft connection element is arranged at a proximal end of the accessory shaft and the guide element is arrangeable to be proximally connected thereto in an assembled state.

13. The connection apparatus according to claim 11, wherein the guide element is configured as part of a monopolar or bipolar radio-frequency connector.

14. The connection apparatus according to claim 13, wherein the third shape-coding dimensions are configured such that a transmission element of a monopolar accessory cannot be inserted into a through-opening of a guide element for bipolar connection.

15. A surgical instrument, for microinvasive surgery, comprising:
a connection apparatus according to comprising an instrument base, a receiving device that is arranged at a shaft opening of the instrument base and comprises a first shape-coding dimension and a second shape-coding dimension, a shaft connection element which is configured to connect an accessory shaft to the instrument base, wherein the shaft connection element is configured as a hollow body through which a transmission element is guided in the accessory shaft, wherein the shaft connection element also has a first shape-coding dimension and a second shape-coding dimension, wherein the first and second shape-coding dimensions are dimensioned such that they correspond to the first and second shape-coding dimensions of the receiving device when the shaft connection element is coupled to the instrument base in an assembled state, and such that the shaft connection element cannot be assembled with the instrument base if the first and/or second shape-coding dimensions of the shaft connection element and the receiving device do not correspond, wherein the instrument base can be operated currentless or in a monopolar or bipolar manner; and
an accessory that can be operated currentless or in a monopolar or bipolar manner and can be mechanically coupled to the connection apparatus.

16. The connection apparatus according to claim 1, wherein the transmission element comprises pull rod guided in the accessory shaft.

17. The connection apparatus according to claim 9, wherein the first shape-coding dimensions are configured such that a shaft connection element of an accessory shaft of a bipolar accessory cannot be inserted into a receiving device for currentless or monopolar connection as a result of an outer diameter of the shaft connection element that is larger than the inner diameter of the receiving device.

18. The connection apparatus according to claim 10, wherein the second shape-coding dimensions are configured such that a shaft connection element of an accessory shaft of a currentless accessory cannot be completely inserted into a receiving device for monopolar or bipolar connection as a result of a length between a proximal end and a projection of the shaft connection element provided for latching purposes being larger than a distance provided between a stop provided in the instrument base and a latching means that is integrated in the receiving device.

19. The connection apparatus according to claim 13, wherein the guide element is configured as part of a monopolar or bipolar radio-frequency connector as a pre-assembled plug-in module for modular installation and removal in and from an instrument base.

20. The connection apparatus according to claim 14, wherein the third shape-coding dimensions are configured such that a transmission element of a monopolar accessory cannot be inserted into a through-opening of a guide element for bipolar connection as a result of an outer diameter of the transmission element which is larger than the diameter of the opening.
